(19) United States
(12) Patent Application Publication
(10) Pub. No.: US 2017/0128360 A1
(43) Pub. Date: May 11, 2017

(54) COMPOSITIONS AND METHODS FOR TREATING CANCER

(71) Applicant: Purdue Research Foundation, West Lafayette, IN (US)

(72) Inventor: Ruben Claudio Aguilar, West Lafayette, IN (US)

(21) Appl. No.: 15/319,386

(22) PCT Filed: Jun. 16, 2015
(86) PCT No.: PCT/US15/36108

§ 371 Date: Dec. 16, 2016

Related U.S. Application Data
(60) Provisional application No. 62/012,622, filed on Jun. 16, 2014.

(51) Int. Cl. A61K 9/00; A61K 38/16; A61K 38/45; A61K 38/18

(52) U.S. Cl. CPC A61K 9/0034; A61K 38/1808; A61K 38/164; A61K 38/45; C12Y 204/02036

(57) ABSTRACT

The invention generally relates to compositions and methods for treating cancer. In certain embodiments, the invention provides methods that involve treating a cancer in a patient in which cancerous cells overexpress epidermal growth factor receptor as compared to non-cancerous cells. The methods involve administering a first composition including anthrax protective antigen modified to bind an epidermal growth factor receptor of a cell, and administering a second composition including anthrax lethal factor N-terminus fused to a catalytic domain of Diphtheria Toxin A. Binding of anthrax lethal factor N-terminus to anthrax protective antigen results in internalization of Diphtheria Toxin A into the cancerous cell, which triggers apoptosis by inactivation of critical elongation factors.

This application claims priority to U.S. Provisional Patent Application 62/012,622 filed on Jun. 16, 2014, which is incorporated herein in its entirety.

### FIELD OF THE INVENTION

The invention generally relates to compositions and methods for treating cancer.

### BACKGROUND

Bladder cancer is the 4th most prevalent cancer in men and the 11th in women. Despite its impact on human health, therapeutic approaches against this malignancy are limited.

Under normal conditions, bladder epithelial cells assemble as a tightly sealed, non-permeable barrier bearing a thick layer of glycosaminoglycans (GAG) that greatly contributes to isolate the urothelium from the urine. However, upon carcinoma development, relatively undifferentiated tumor cells, less competent for secretion, become exposed while normal cells remain shielded by the GAG layer. Those uncovered tumor cells are suitable targets for cytotoxic agents. However, dilution of the bladder content by constant urine influx and periodical voiding constitute major challenges for therapeutic approaches with poor or non-existing cell binding/targeting.

### SUMMARY

The invention recognizes that in contrast to normal cells, bladder tumor cells are exposed to the bladder lumen and they express high levels of epidermal growth factor receptor (EGFR). Accordingly, the invention provides therapeutic compositions modified to include epidermal growth factor (EGF), thereby targeting the claimed compositions to exposed cancerous cells along the bladder lumen that overexpress EGFR. In particular embodiments, this approach uses the highly efficient and fast EGF-induced internalization of EGFR to deliver a lethal bacterial toxin into the cytosol of exposed tumor cells. The data herein show that under bladder instillation conditions, the claimed compositions targeted human and mouse bladder cancer cells with a LC₁₀₀ of less than 1 nM and an exposure time of less than 3 min. Due to the ability of the claimed compositions to target superficial and invasive cancer, its high efficiency and quick action (current treatments require ˜2 h retention of therapeutics in the bladder), the claimed compositions are effective for treating cancers that overexpress EGFR, such bladder cancer.

### BRIEF DESCRIPTION OF THE DRAWINGS

FIG. 1A shows a scheme of the bladder and bladder urothelium architecture;

FIG. 1B shows the putative mechanism of action of the EGF-toxin;

FIG. 1C shows that human bladder cells can be targeted by EGF;

FIG. 1D shows fast targeting and elimination of human bladder cancer cells by EGF-toxin;

FIG. 2A shows EGF-toxin binding and specificity;

FIG. 2C shows kinetics of toxin action;

FIG. 2D shows proposed treatment-like conditions;

FIG. 3A shows a workflow diagram for bladder cancer patient samples;

FIG. 3B shows bladder cancer patient cells binding fluorescent (tetra-methyl-rhodamine: TMR)-EGF as detected by epifluorescence microscopy;

FIG. 3C shows bladder cancer patient cells binding fluorescent (tetra-methyl-rhodamine: TMR)-EGF as detected by flow-cytometry;

FIG. 4A shows the binding of EGF to mouse bladder cancer cells;

FIG. 4B shows elimination of MB49 bladder cancer cells by EGF-toxin;

FIG. 4C shows a mouse orthotopic model of bladder cancer; and

FIG. 5 shows that EGF-toxin targets and eliminates canine invasive bladder cancer cells.

### DETAILED DESCRIPTION

Aspects of the invention are based on the recognition that bladder tumor cells are highly sensitive to the claimed compositions due to their high levels of fast internalizing EGFR. It is believed that the claimed compositions provide for an innovative and highly efficient anti-bladder cancer strategy using a new generation of targeted agents. The claimed compositions are a superior agent due to their high efficacy and fast action, but also due to their ability to target superficial and invasive bladder cancers (both rich in EGFR levels). Further, the impact of this development reaches beyond the realm of bladder cancer, and is applicable to any cancer in which EGFR is overexpressed, such as lung and skin cancers.

Since both superficial and invasive bladder carcinomas are known to overexpress Epidermal Growth Factor (EGF) Receptor (EGFR), it is believed that an EGF targeted therapeutic composition, such as an EGF-targeted lethal toxin (referred to as the EGFR binding moiety-toxin system or just EGF-toxin), would be transformative in bladder cancer therapeutics.

In one aspect of the present invention there is provided an EGFR binding moiety-toxin system or just EGF-toxin system comprising two components. The two components may comprise a first component having an EGFR binding moiety linked to an anthrax protective antigen. The first component may be expressed as a fusion protein. Alternatively, the first component may have the EGFR binding moiety chemically linked to the anthrax protective antigen. The EGFR binding moiety may be EGF or fragments of EGF known to bind to the EGFR receptor. The EGFR binding moiety alternatively may be a compound known in the art to, or specifically designed to bind to EGFR. Examples of such EGFR binding moieties are known to the skilled artisan. For example, U.S. Pat. No. 6,941,229, incorporated herein in its entirety, gives examples of EGFR binding moieties as well as methods for designing such moieties.

In another aspect of the present invention, the second component may comprise the toxin component of the system. The second component may be an anthrax lethal factor N-terminus linked to a catalytic domain of diphtheria toxin. The second component may be expressed as a fusion protein. Alternatively, the second component may have the anthrax lethal factor chemically linked to the catalytic domain of diphtheria toxin. It will be apparent to the skilled artisan that other toxins may be substituted for the anthrax lethal factor and the diphtheria toxin. While no wishing to be bound by theory it is believed that the first component of the system binds to EGFR on the surface of the bladder cancer cell, activating the transport mechanism of the receptor. The presence of the anthrax protective antigen allows for the binding of the toxin to the first component, thus allowing it to be transported into the cell and ultimately killing the cell. Therefore the skilled artisan would have the knowledge to substitute the antigen and toxin described herein with another pair without undue experimentation.

The present invention also provides methods for using the system of the present invention for treating bladder cancer in a mammal. The EGF-toxin system may be applied directly to the bladder cancer through a catheter. In one embodiment the method comprises the steps of administering a therapeutic amount of the first component, waiting a set amount of time to allow the first component to bind to EGFR on the bladder cancer cells and then administering a therapeutic amount of the second component. As a non-limiting example, a therapeutic amount of the first component may be from about 0.1 nM to about 10 nM or, alternatively, from about 0.5 nM to about 6.0 nM. As a non-limiting example, a therapeutic amount of the second component may be from about 1 nM to about 20 nM or, alternatively, from about 5 nM to about 15 nM. The set amount of time to allow the first component to bind to EFGR may be from about 1 minute to about 30 minutes or alternatively, from about 3 minutes to about 15 minutes.

In an alternate embodiment, the method of the present invention may comprise administering a therapeutic dose of both the first component and the second component of the EGF-toxin system simultaneously. As a non-limiting example, a therapeutic amount of the first component may be from about 0.1 nM to about 10 nM or, alternatively, from about 0.5 nM to about 6.0 nM. As a non-limiting example, a therapeutic amount of the second component may be from about 1 nM to about 20 nM or, alternatively, from about 5 nM to about 15 nM.

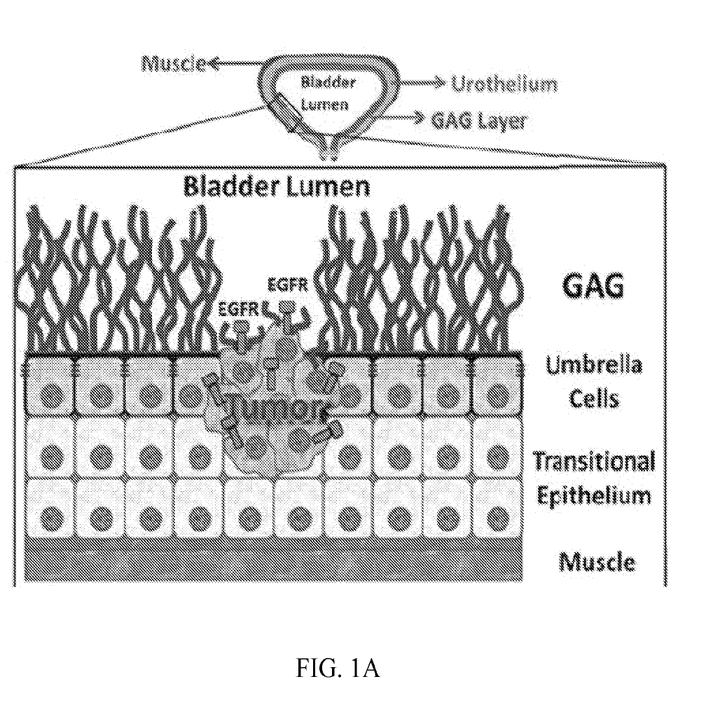

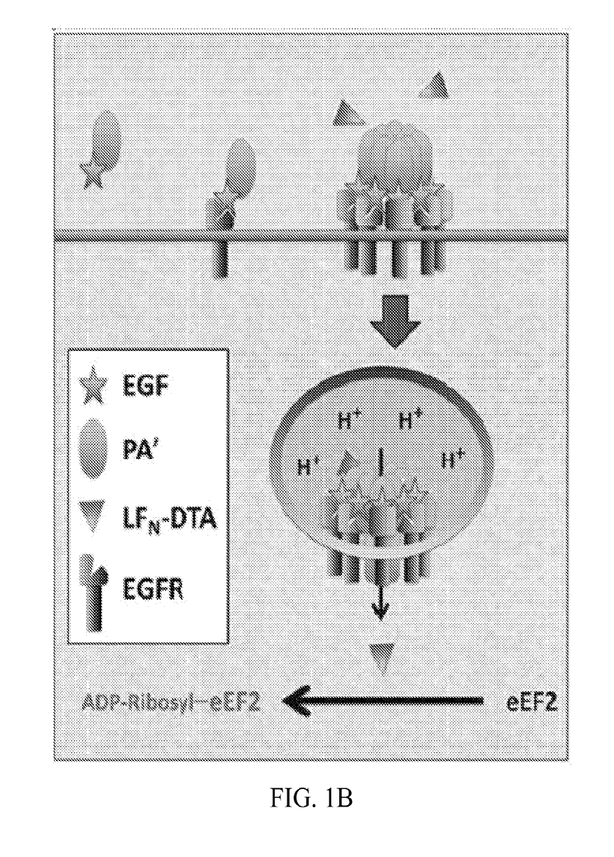

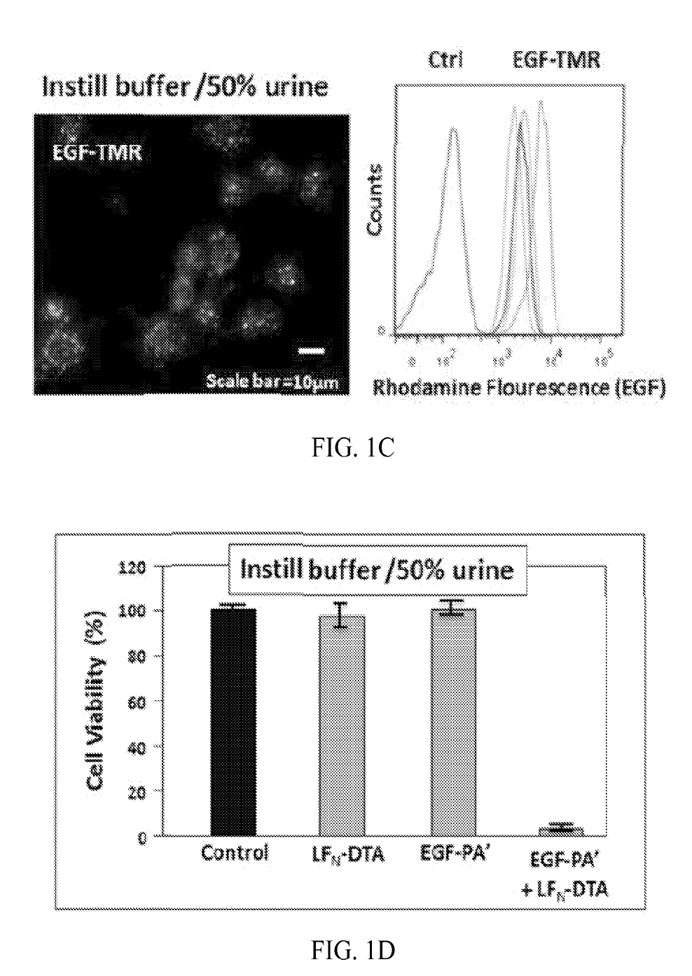

The EGF-toxin system and its mechanism of action is shown in FIG. 1. Briefly, an EGF-anthrax Protective Antigen fusion protein (EGF-PA′) binds to EGFR, assembles as an octameric pre-pore complex on the plasma membrane and recruits 2-3 molecules of LF_{N}-DTA (anthrax Lethal Factor N-terminus fused to the catalytic domain of Diphtheria Toxin A; FIG. 1B). After clathrin-mediated endocytosis, the lower pH of the endosome induces a conformational change in the EGF-PA′ complex leading to pore formation and translocation of LFN-DTA molecules, which in turn trigger apoptosis by inactivation of critical elongation factors (FIG. 1B). It should be noted that the binary nature of the toxin, is a safety-guard against potential leaks into the bloodstream (as dilution greatly decreases the probability of toxin reconstitution on normal cells). The data herein indicates that the EGF-toxin can efficiently target and eliminate human, mouse and canine bladder tumor cells (see FIGS. 1-4), offering a novel, fast and efficacious strategy against both superficial and invasive bladder cancer.

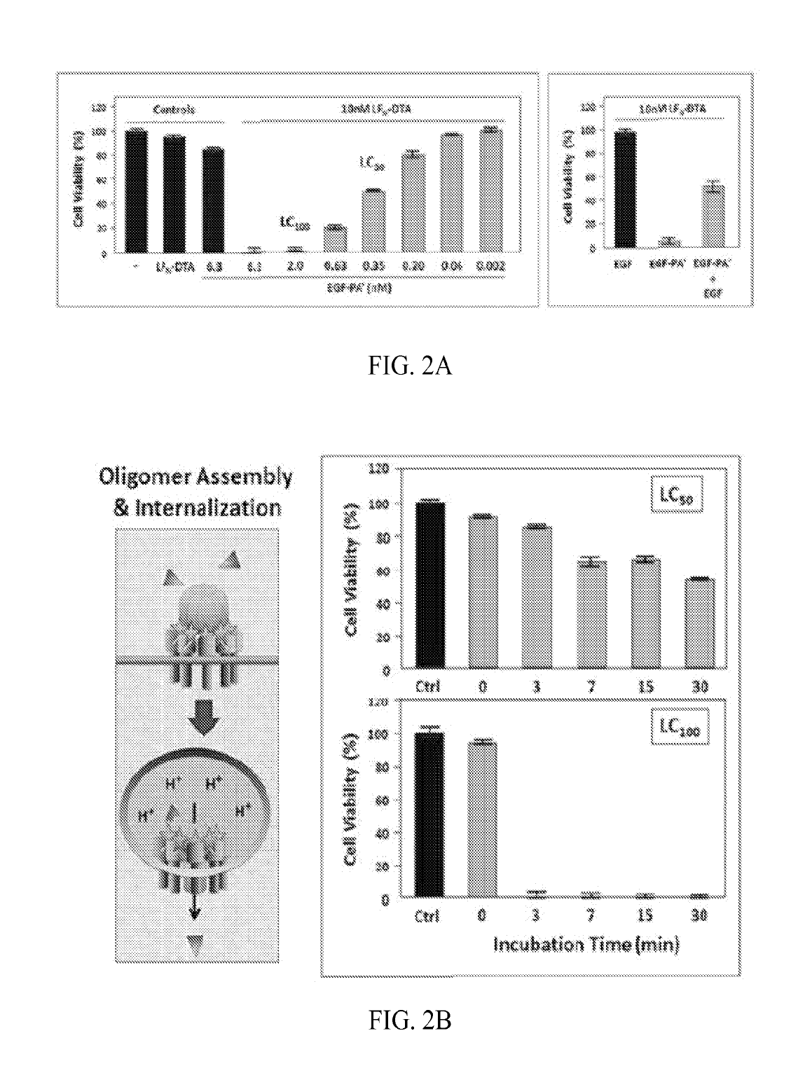

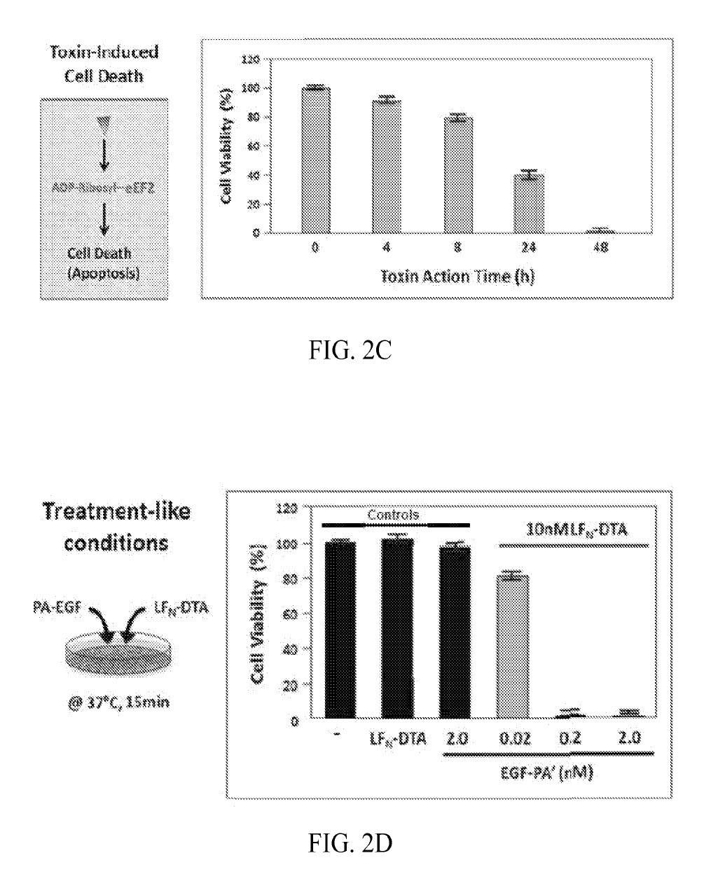

FIGS. 1C, 3 and 4A show that EGF is capable of targeting human and mouse bladder cancer cells (inferred from binding of fluorescently-labeled EGF detected by imaging and flow cytometry). The EGF-toxin was highly efficient for the elimination of human cancer cells under conditions that emulate the environment of the bladder during treatment (i.e., instillation buffer +50% human urine; FIG. 1D). Next, an experimental approach was designed to dissect 3 different steps of toxin action (FIG. 2 and legend therein): binding (FIG. 2A), octamer assembly/internalization/LF_{N}-DTA translocation (FIG. 2B) and LF_{N}-DTA action (FIG. 2C). Using that set up, the EGF specificity of the EGF-toxin was established (FIG. 2A, right) and its LC₅₀ (0.35 nM) and LC₁₀₀ (2 nM) (FIG. 2A, left) were determined. By fixing the EGF-PA′ concentration to its LC₅₀ value the time-dependence was determined for the assembly/internalization/translocation phase of the EGF-toxin action (FIG. 2B, upper panel).

By using EGF-PA′ at its absolute lethal concentration (LC₁₀₀), it was established that times as short as 3 min. assured maximal killing by the toxin. That data illustrates that this novel strategy represents a breakthrough in bladder cancer therapies due to its high efficiency, and also drastically decreases patient treatment time from hours (current therapies) to a few minutes. In addition, although it was established that the first apoptosis indicators (fluorescent annexin-V binding) are detectable after 4 h of exposure to the toxin, 100% cell death occurred 48 h after toxin action (FIG. 2C). As expected, “treatment-like” conditions (i.e., simultaneous exposure of the cells to EGF-PA′/LF_{N}-DTA in instillation buffer/urine, at 37° C. and without elimination of unbound or non-internalized proteins) led to an increase in the sensitivity of the cells to the toxin, observed as 10× decrease in the LC₁₀₀ (from 2 nM to 0.2 nM—FIG. 2D). Taken together, the data illustrates the high translational ability of the EGF-toxin as a superior therapeutic agent against bladder cancer (Table I)

TABLE I
Therapeutic Agent Comparison

| | FAP-Nanoparticle | EGF-Toxin |
|---|---|---|
| 1. Stability | hours | years |
| 2. Affinity | Micromolar? | Sub-nanomolar |
| 3. Rate of Action | 2 h | 3 min |
| 4. Cytotoxicity | LC₅₀ ~5-10 μM | LC₁₀₀ <1 nM |
| 5. Predicted | Particle level | Molecular level |
| Tumor penetration | (~50 nm and | (Å) |
| | Heterogeneous) | |

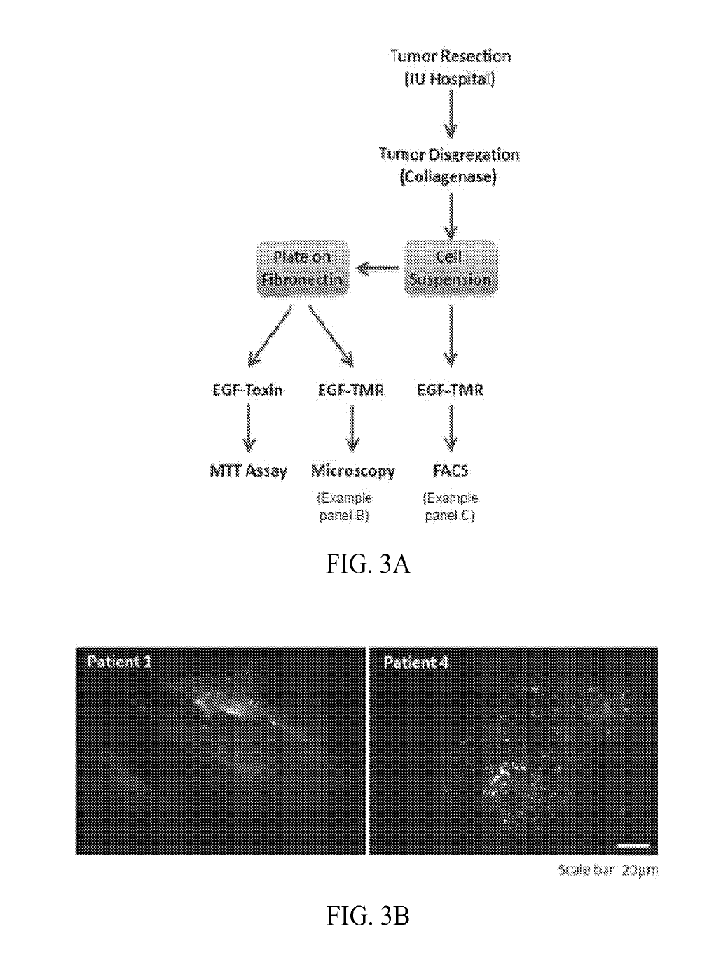

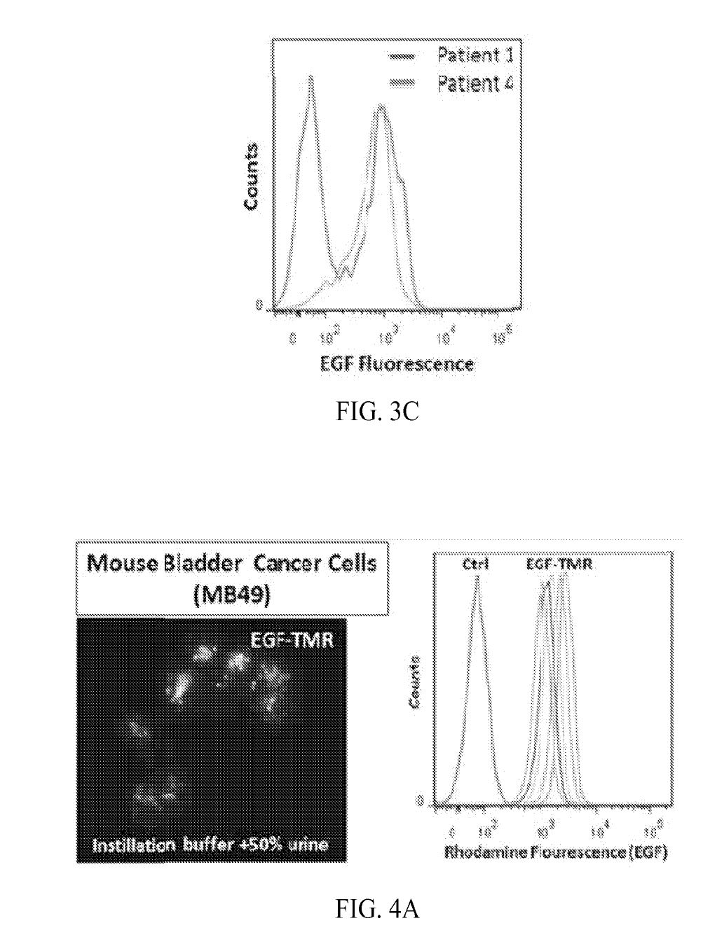

The effect of naturally-occurring EGFR level heterogeneities within and between patient tumors on the EGF-toxin efficacy is determined. Cells from bladder tumors freshly resected from patients at Indiana University (FIG. 3) are used, and normal human bladder epithelial primary cells (Lifeline Cell Tech., FC-0079) are a control.

One-two grams of resected primary human bladder cancer tissue is obtained. The tissue is minced into approximately 1 mm×1 mm pieces and digested with collagenase+DNAse for 1.5 h with shaking at 37° C. and purified/enriched in viable cells by low-speed centrifugation using standard approaches to produce a tumor cell suspension. Part of the cells in suspension are seeded on fibronectin-coated surfaces (FIG. 3A) (fibronectin is a typical component of the extracellular matrix in bladder tumors). These adherent cells are used to test EGF-toxin killing efficacy (MTT assays) as in FIG. 2 (i.e., to determine toxin LC₅₀ and rate of action in the presence or absence of excess unmodified EGF). They are also used to measure fluorescent EGF-TMR binding as in FIG. 1C. The data obtained supports the existence of EGF binding heterogeneity within (FIG. 3B; patient 1) and in-between tumors (FIG. 3B; patient 1 vs. 4). The remaining suspension of cells are used for FACS analysis following binding of EGF-TMR (FIG. 3A). Preliminary flow cytometry data also indicates EGF-binding heterogeneities within/in-between tumors (FIG. 3C and legend therein). EGFR in whole cell lysates are also detected by Western blotting with specific antibodies. The results are quantified by band densitometry.

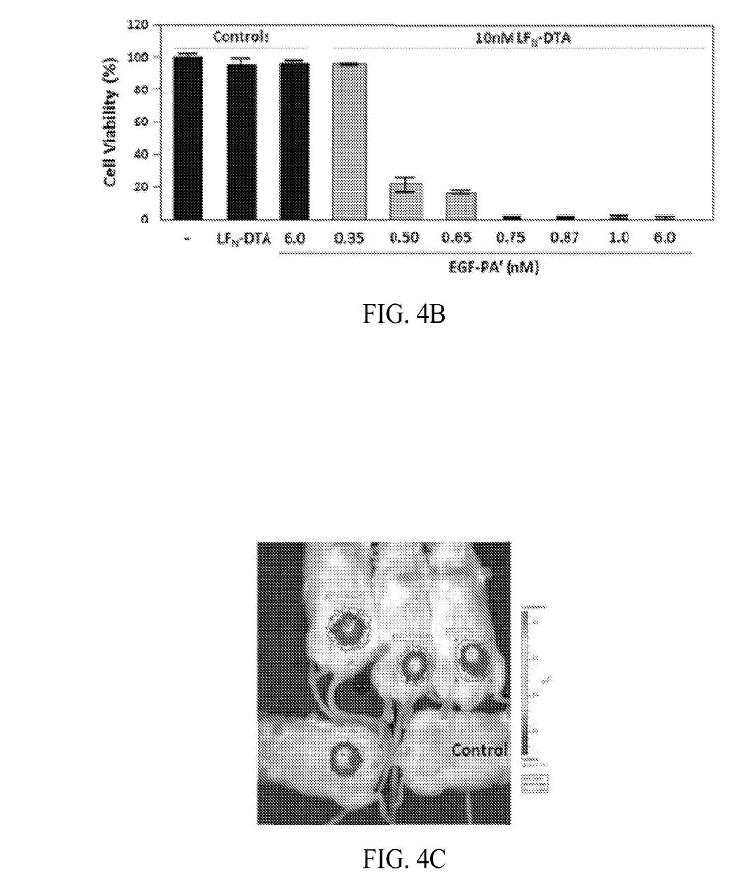

The ability of EGF-toxin to eliminate mouse bladder cancer cells expressing different levels and variants of EGFR are also determined. The mouse bladder cancer cell line MB49 is suitable for in vitro manipulation of EGFR levels allowing to test toxin-sensitivity as function of the levels and nature of EGFR variants. Data herein (FIG. 4) show that similar to human T24 bladder cancer cells (FIGS. 1-2), MB49 cells can be targeted and eliminated (in a dose-dependent manner) by the EGF-toxin. The MB49 cell line is chosen (over T24) for this purpose due to consistency reasons, as these cells are used for the establishment of an orthotopic bladder cancer model (discussed below). Therefore, MB49 cells (WT cell line) are used, EGFR-deficient (shRNA-mediated knock-down), or overexpressing EGFR^{WT} and EGFR^{K721M} (internalization deficient mutant) to perform the same experimental determinations as above. Specifically, toxin LC₅₀ and rate of action (+/−excess of unmodified EGF) were determined, optimal LF_{N}-DTA concentration were titrated; and EGF binding and EGFR levels were measured.

It is expected that cells with low levels of EGFR (naturally occurring in patients/generated by knock-down in MB49) or expressing the EGFR^{K721M} internalization mutant are less sensitive (higher LC₅₀) to the EGF-toxin effects than EGFR^{WT} overexpressors.

The ability of EGF-toxin to reduce tumor growth in a bladder cancer mouse orthotopic model based on MB49 cells expressing different levels of EGFR were determine in vivo. Orthotopic bladder tumors were generated in C57BL/6 mice. Briefly, mice were anesthetized with a 90/10 mg/kg ketamine/xylazine mixture and then catheterized by inserting a 24-gauge i.v. catheter through the urethra and into the bladder lumen. The bladder was prepared for site-specific tumor adherence with electrocautery. Under anesthesia, an electrode of 4-0 surgical stainless steel wire was inserted through the catheter far enough to contact the bladder wall. The electrode was attached to a Bovie electrocautery unit which was activated for 4 sec. at the lowest coagulation setting. The electrode was removed and bladders were instilled with 10⁵ MB49 cells stably expressing luciferase in 100 μl of RPMI medium. Intravesical tumor growth was monitored daily by luciferase imaging following intraperitoneal injection of luciferin salt (15 mg/kg) using an IVIS Lumina II imaging equipment (FIG. 3C); and by ultrasound with a VisualSonics 2100 ultrasound system specifically designed to monitor tumor volume in anesthetized mice. A chemical depilatory cream was used to remove abdominal hair, followed by application of Aquasonic ultrasound gel to minimize reflections between the transducer and the skin. Tumor bearing mice were randomly assigned to five groups according to Table II.

TABLE II

| | | Time of | | Purpose of | Additional |
|---|---|---|---|---|---|
| Group^{a} | Treatment^{b} | Treatment^{c} | | the Group | Treatment |
| Control 1 | Instillation | ~3-4 | days^{d} | Controls | EGF- |
| | Buffer | | | | TMR^{f} |
| Control 2 | EGF-PA′ | | | | None |
| Control 3 | LF_{N}-DTA | | | | |
| Treatment 1 | EGF-PA′ + | | | Tumor growth | |
| | LF_{N}-DTA | | | prevention | |
| Treatment 2 | EGF-PA′ + | ~1 | week^{e} | Tumor growth | |
| | LF_{N}-DTA | | | reduction | |

^{a}5mice/group.
^{b}EGF-PA′ and LF_{N}-DTA (in instiliation buffer) will be applied at 2 nM and 10 nM final concentration, respectively.
^{c}Time post-tumor implantation.
^{d}Estimated time of early tumor detection by bioluminescence.
^{e}Estimated time of development of 200 mm³ tumor as detected by ultrasound.
^{f}Following euthanasia tumors from “Control 1” group will be isolated and incubated with 2 nM fluorescent EGF for 30 min, washed thrice and processed for imaging.

Bladders were catheterized and subjected to 100 μl instillations of the indicated solutions (“Treatment” column, Table II), allowed to dwell for 30 min. and washed three times with fresh buffer. All surviving mice were euthanized after 2 weeks and the bladders weighed and examined grossly for the presence of tumors and subsequently by histology. Experiments were repeated at least 3 times. Bladder tumors were isolated from mice belonging to control group 1 and incubated with 2 nM fluorescent EGF-TMR for 30 min. in instillation buffer, washed thrice and processed for imaging. This procedure provided preliminary data on tumor penetration by EGF. For the animal experiments, sample size was calculated using standard power analysis to 5 mice/experimental group with a α of 0.05 and a power of 80%. For the present application, and since the typical orthotopic implantation rate was >90%, the number of mice was increased to 6/group. 30 mice (3 controls and 2 treatments—Table II) were used per experiment and each experiment was repeated thrice yielding 90 mice (30 mice×3). In addition, maximal orthotopic implantation rate was achieved using in vivo passaged MB49 tumor cells. Typical subcutaneous (SQ) implantation rate of in vitro cultured MB49 cells was approximately 60-80% (3 out of 4 mice); therefore, 4 mice were implanted with MB49 SQ to provide the 3 tumor-bearing mice to ensure sufficient viable cells for the experiments. Therefore, 4 mice×the 3 individual experiments=12 mice. Overall, 102 mice (90 experimental+12 donor) were used for the proposed experiments. The Table II experimental set-up was repeated using MB49 stably-overexpressing EGFR^{WT} for tumor generation (yielding a total of 204 mice). Statistical analysis was performed using two-way ANOVA. Toxin-treated animals showed diminished sized tumors or were tumor free.

In certain embodiments, compositions of the invention are formulated with a pharmaceutically acceptable carrier. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

In certain embodiments, compositions of the invention are pegylated. As used herein the term “pegylated” and like terms refers to a compound that has been modified from its native state by linking a polyethylene glycol polymer to the compound. As used herein the general term “polyethylene glycol” or “PEG”, refers to mixtures of condensation polymers of ethylene oxide and water, in a branched or straight chain, represented by the general formula H(OCH₂CH₂)ₙOH, wherein n is at least 9. Absent any further characterization, the term is intended to include polymers of ethylene glycol with an average total molecular weight selected from the range of 500 to 40,000 Daltons. “polyethylene glycol” or “PEG” is used in combination with a numeric suffix to indicate the approximate average molecular weight thereof. For example, PEG-5,000 refers to polyethylene glycol having a total molecular weight average of about 5,000.

It has been found that compositions of the invention enhance the uptake diagnostic and therapeutic agents by mammalian cells, including for example bladder cells, particularly bladder tumor cells of cancer patients. Particularly, the multivalent nature of the compositions of the invention imparts improved binding affinity and rate of uptake as compared to microcluster compositions. In addition, the compositions of the invention have superior protein solubility/stability as compared to monovalent compositions that are tied together into a microcluster.

Accordingly, the invention also provides methods for treating a cancer in which cancerous cells express a moiety that may be bound by compositions of the invention. The compositions of the invention can be formulated in a pharmaceutically acceptable carrier and administered to the lumen of the bladder using standard techniques known to those skilled in the art. In one embodiment the pharmaceutical composition is delivered by direct administration (via injection or by catheterization) of the composition into the bladder lumen. The pharmaceutical compositions can be further packaged as part of a kit that includes a disposable device for administering the composition to a patient. In one embodiment the kit is provided with a device for administering the composition to a patient. The kit may further include a variety of containers, e.g., vials, tubes, bottles, and the like. The kits may also include instructions for use.

In one embodiment the composition is formulated in association with a liposome, in which claimed peptide composition is presented on the external surface of the liposome and the diagnostic agent or therapeutic agent is encapsulated within the liposome. Since within the bladder, the claimed composition specifically binds to targets only exposed on bladder tumors (normal cells are shielded by the GAG layer), the claimed compositions can be used as an intelligent drug carriers capable of selective delivery of a drug (such as an anti-tumor agent). If a composition of the invention is further linked with a conventional anti-tumor agent, it is possible to increase the efficacy of the anti-tumor agent and significantly reduce side effects adversely affecting normal tissue because the anti-tumor agent is delivered selectively to a bladder tumor cell by the compositions disclosed herein. In accordance with one embodiment the claimed composition/anti-tumor complexes can be further provided with additional cancer targeting moieties (e.g., anti-tumor antibodies) to further target the complexes to cancer cells. In one embodiment the composition is delivered by direct administration (via injection or by catheterization) of the composition into the bladder lumen.

In addition, the composition according to the present invention may further comprise pharmaceutically acceptable carriers that are added conventionally to a general pharmaceutical composition. In the case of injection formulation, particular examples of the pharmaceutically acceptable carriers include a buffering agent, a preserving agent, an anesthetic agent, a solubilizing agent, an isotonic agent and a stabilizer. The composition can as a unit dose ample or a multidose vial.

A “liposome” as used herein refers to a small, spherical vesicle composed of lipids, particularly vesicle-forming lipids capable of spontaneously arranging into lipid bilayer structures in water with its hydrophobic moiety in contact with the interior, hydrophobic region of the bilayer membrane, and its head group moiety oriented toward the exterior, polar surface of the membrane. Vesicle-forming lipids have typically two hydrocarbon chains, particularly acyl chains, and a head group, either polar or nonpolar. Vesicle-forming lipids are either composed of naturally-occurring lipids or of synthetic origin, including the phospholipids, such as phosphatidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation. The above-described lipids and phospholipids whose acyl chains have varying degrees of saturation can be obtained commercially or prepared according to published methods. Other suitable lipids for use in the composition of the present invention include glycolipids and sterols such as cholesterol and its various analogs which can also be used in the liposomes.

Cationic lipids, which typically have a lipophilic moiety, such as a sterol, an acyl or diacyl chain, and where the lipid has an overall net positive charge can also be suitably used in liposomes. The head group of the lipid typically carries the positive charge. Exemplary cationic lipids include 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1 -(2,3,-dioleyloxy)propyl]-N,N-dimethyl-N-hydroxy ethylammonium bromide (DORIE); N-[1-(2,3-dioleyloxy)propyl]-N,N,N-trimethylammonium chloride (DOTMA); 3 [N˜(N′,N′-dimethylaminoethane) carbamolyjcholesterol (DC-Choi); and dimethyldioctadecylammonium (DDAB). The cationic vesicle-forming lipid may also be a neutral lipid, such as dioleoylphosphatidyl ethanolamine (DOPE) or an amphipathic lipid, such as a phospholipid, derivatized with a cationic lipid, such as polylysine or other polyamine lipids.

The liposomes can include a vesicle-forming lipid derivatized with a hydrophilic polymer to form a surface coating of hydrophilic polymer chains on the liposomes surface. A vesicle-forming lipid, in particular a phospholipid, such as distearoyl phosphatidylethanolamine (DSPE), may be covalently attached to a hydrophilic polymer, which forms a surface coating of hydrophilic polymer chains around the liposome. Hydrophilic polymers suitable for derivatization with a vesicle-forming lipid include polyvinylpyrrolidone, polyvmylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyemylcellulose, polyethyleneglycol, polyaspartamide and hydrophilic peptide sequences. The polymers may be employed as homopolymers or as block or random copolymers.

One hydrophilic polymer chain suitable for use is polyethyleneglycol (PEG), preferably as a PEG chain having a molecular weight between 200-20,000 daltons, or between 500-10,000 daltons, or between 750-5000 daltons. Methoxy or ethoxy-capped analogues of PEG are also preferred hydrophilic polymers, commercially available in a variety of polymer sizes, e.g., 120-20,000 Daltons. In one embodiment the PEG polymers are derivatized (e.g. at the free end) to further comprise a ligand that binds to a fibronectin attachment protein.

Preparation of Vesicle-Forming Lipids Derivatized with Hydrophilic Polymers has been described, for example in U.S. Pat. No. 5,395,619, in U.S. Pat. No. 5,013,556, in U.S. Pat. No. 5,631,018 and in WO 98/07409. It will be appreciated that the hydrophilic polymer may be stably coupled to the lipid, or coupled through an unstable linkage, which allows the coated liposomes to shed the coating of polymer chains as they circulate in the bloodstream or in response to a stimulus. In one embodiment the liposomes are derivatized to include a plurality of antibodies or ligands that specifically bind to a fibronectin attachment protein.

### EXAMPLES

### Example 1

### Claimed Compositions Target Human Cancer Cells

FIGS. 1A-D shows that the claimed compositions (also referred to as EGF-toxin) targets and eliminates human bladder cancer cells. FIG. 1A shows a scheme of the bladder and bladder urothelium architecture. In contrast to normal differentiated umbrella cells, bladder tumor cells are known to overexpress EGFR, be deficient for GAG layer synthesis and tight junction assembly. Therefore tumor cells are exposed to the lumen of the bladder.

FIG. 1B shows the proposed mechanism of action of the EGF-toxin. The specificity of a mutated version of the anthrax protective antigen (PA′; blue oval—unable to recognize the anthrax receptor), is redirected to EGFR by fusion to EGF (star). Following EGFR binding, EGF-PA′ assembles as an octomer on the plasma membrane. That complex is bound by the anthrax lethal factor N-terminus (LF_{N}) fused to the catalytic domain of diphtheria toxin (DTA—the LF_{N}-DTA fusion is represented as a green triangle). Following internalization, the lower pH of the endosome induces a conformational change in the EGF-PA′ octomer that promotes its insertion into the endosomal membrane and LF_{N}-DTA translocation into the cytosol. LF_{N}-DTA catalyzes the ADP-ribosylation of Eukaryotic elongation factor 2 (eEF2).

FIG. 1C show that human bladder cells can be targeted by EGF. Fluorescent (tetra-methyl-rhodamine: TMR)-EGF was bound and internalized by human T24 bladder cancer cells in the presence of instillation buffer and 50% urine, as detected by epifluorescence microscopy (left) and flow cytometry (right—different colors under EGF-TMR label represent different ligand concentrations).

FIG. 1D shows fast targeting and elimination of human bladder cancer cells by EGF-toxin. 20,000 T24 cells were plated per well on 6-well plates and incubated for 8 min. in presence or absence (“control”) of the indicated reagents in instillation buffer supplemented with 50% human urine. After 48 h cell viability was measured by MTT assays and represented as % of the control of triplicates.

### Example 2

### Characterization and Optimization of the EGF-Toxin Targeting and Elimination of Human Bladder Cancer Cells

FIGS. 2A-D show characterization and optimization of the EGF-Toxin targeting and elimination of human bladder cancer cells. To characterize and optimize binding, internalization and toxin action time (FIGS. 2A-C), a stepwise approach was designed: 2×10⁴ serum-“starved” T24 cells (MTT linear range) are plated and incubated with the indicated concentration of EGF-PA′ for 45 min on ice to prevent uptake. Following washes to eliminate unbound ligand, the cells are incubated with 10 nM LF_{N}-DTA at 37° C. for the indicated times to allow octamer assembly, internalization and LF_{N}-DTA translocation. Next, non-internalized complexes are stripped off with acidic washes, complete media is added and the cells are kept at 37° C. for the indicated amount of time before MTT assays are conducted.

FIG. 2A shows EGF-toxin binding and specificity: Drug-response relationship. Left: Experimental setup described above was performed incubating serum-“starved” T24 cells with different concentrations of EGF-PA′ for 45 min on ice. Other experimental variables were fixed: incubation with LF_{N}-DTA at 37° C. was conducted for 30 min, and MTT assays were performed 48 h after toxin exposure. Absolute Lethal Concentration (LC₁₀₀) and Lethal Concentration 50% (LC₅₀) were determined. Right: In order to test the EGFR specificity of the EGF-toxin, a 50× excess of unmodified EGF was added to compete EGF-PA′ binding to the receptor.

FIG. 2B shows kinetics of toxin assembly and Internalization. Experimental setup was conducted by binding EGF-PA′ at either LC₅₀ (top) or LC₁₀₀ (bottom) concentrations on ice, and allowing complex assembly and internalization for different times before stripping off non-intermalized protein and adding complete media. MTT assays were conducted 48 h later.

FIG. 2C shows kinetics of toxin action. Cells were incubated with LC₁₀₀ concentration of EGF-PA′ on ice, followed by LF_{N}-DTA at 37° C. for 30 min to allow assembly and uptake of the toxin. MTT assays were performed at the indicated times to monitor cell viability as a function of time.

FIG. 2D shows “Treatment-like” conditions. In contrast to the stepwise approach, EGF-PA′ (at the indicated concentrations) and 10 nM LF_{N}-DTA were added to the cells simultaneously at 37° C. for 30 min and without elimination of unbound or non-internalized toxin.

### Example 3

### Bladder Cancer Patient Cells Bind Fluorescent (Tetra-Methyl-Rhodamine: TMR)-EGF

FIG. 3A shows a workflow Diagram for bladder cancer patient samples. FIGS. 3B-C show bladder cancer patient cells bind fluorescent (tetra-methyl-rhodamine: TMR)-EGF as detected by epifluorescence microscopy (FIG. 3B) and flow-cytometry (FIG. 3C). Note the EGF-binding heterogeneities within patient 1 tumor cells: FIG. 3B on the left shows a cell with substantial EGF-TMR binding (cell above) accompanied by two other with lower binding capacity. A similar result was obtained by FACS analysis, patient 1 exhibited 2 populations of cells: one with substantial EGF-TMR binding capacity than the other (red line peaks). Patient 4 cells appear more homogeneous with high levels of EGF-TMR binding.

### Example 4

### Characterization and Optimization of the EGF-Toxin Targeting and Elimination of Human Bladder Cancer Cells

FIGS. 4A-C show characterization and optimization of the EGF-Toxin targeting and elimination of mouse bladder cancer cells. FIG. 4A shows mouse bladder cancer cells bind EGF. TMR-EGF recognition by MB49 cells was demonstrated by epifluorescence microscopy (left) and flow-cytometry (right) as in FIGS. 1C-D. FIG. 4B shows elimination of MB49 bladder cancer cells by EGF-toxin. Effect of the EGF-toxin on MB49 cell viability was measured by MTT assays as in FIG. 1E. FIG. 4C shows mouse orthotopic model of bladder cancer. Implanted bladder tumors were detected in mice bladders by bioluminescence (IVIS imaging) 5 days after instillation of 105 MB49-luc cells (see text for details). Bioluminescence intensity was color-coded according to the scale shown.

### Example 5

### EGF-Toxin Targets and Eliminates Canine Invasive Bladder Cancer Cells

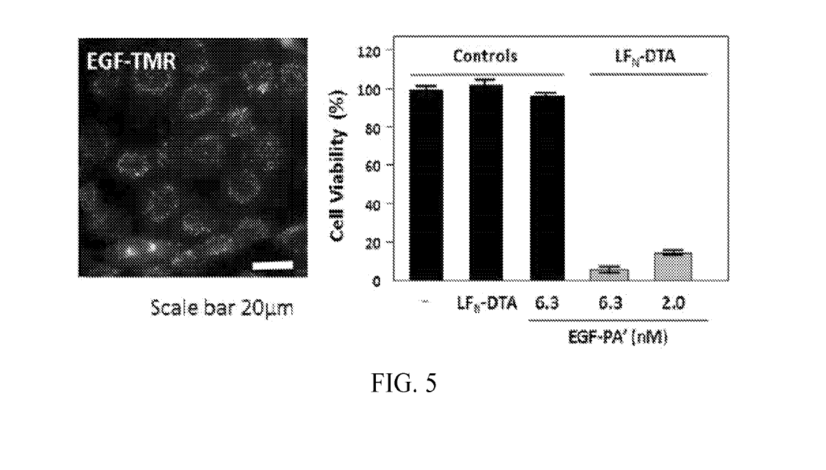

FIG. 5 shows that EGF-toxin targets and eliminates canine invasive bladder cancer cells. Right: Canine invasive bladder cancer cells bind EGF. TMR-EGF recognition was demonstrated by epifluorescenco microscopy. Left: Elimination of Canine invasive bladder cancer cells by EGF-toxin. Effect of the EGF-toxin on cell viability was measured by MTT assays.

1. A composition comprising:
a first component comprising an epidermal growth factor receptor (EGFR) binding moiety linked to an anthrax protective antigen; and
a second component comprising anthrax lethal factor N-terminus linked to a catalytic domain of diphtheria toxin.

2. The composition of claim 1 wherein the EGFR binding moiety is epidermal growth factor (EGF).

3. The composition of claim 1 wherein the first component is a fusion protein of the EGFR binding moiety and the anthrax protective antigen.

4. The composition of claim 1 wherein the second component is a fusion protein of the anthrax lethal factor N-terminus and the catalytic domain of diphtheria toxin.

5. The composition of claim 1 wherein the first component comprises the EGFR binding moiety chemically linked to the anthrax protective antigen.

6. The composition of claim 1 wherein the second component comprises the anthrax lethal factor N-terminus chemically linked to the catalytic domain of diphtheria toxin.

7. A method for treating a cancer in a mammal in which a cancerous cell overexpresses epidermal growth factor receptor (EGFR) as compared to a non-cancerous cell, comprising administering to said mammal a therapeutic dose of the composition of claim 1.

8. The method of claim 7 comprising the steps of:
administering the first component to the mammal in need of relief from said cancer;
waiting a set amount of time; and
administering the second component.

9. The method of claim 7 wherein the first component and the second component are administered simultaneously.

10. The method of claim 7 wherein the composition is administered to the site of a bladder cancer by catherization.

11. A method for treating a cancer in a mammal in which a cancerous cell overexpresses epidermal growth factor receptor (EGFR) as compared to a non-cancerous cell comprising:
administering a first component comprising an anthrax protective antigen modified to bind an EGFR of a cell; and
administering a second component comprising an anthrax lethal factor N-terminus fused to a catalytic domain of diphtheria toxin.

12. The method according to claim 11, wherein the first and the second components are administered sequentially or simultaneously.

13. The method according to claim 11, wherein the first and the second components are formulated into a single pharmaceutical composition and administered as a single composition.

14. The method according to claim 11, wherein the anthrax protective antigen is mutated so that it is unable to bind to an anthrax receptor of a cell.

15. The method according to claim 14, wherein the anthrax protective antigen is a fusion protein with an epidermal growth factor (EGF) domain.

16. The method according to claim 11, wherein binding of anthrax lethal factor N-terminus to an anthrax protective antigen results in internalization of diphtheria toxin into said cancerous cell.

17. The method according to claim 11, wherein said mammal is a human.

18. The method according to claim 11, wherein said cancer is a bladder cancer.

19. The method according to claim 18, wherein administering is by a parenteral route to the site of a bladder cancer by catherization.

20. A kit for treatment of a cancer in a mammal in which a cancerous cell overexpresses epidermal growth factor receptor (EGFR) as compared to a non-cancerous cell comprising:
a first component comprising an anthrax protective antigen modified to bind an EGFR of a cell; and
a second component comprising an anthrax lethal factor N-terminus fused to a catalytic domain of diphtheria toxin.
